(19) United States
(12) Patent Application Publication
(10) Pub. No.: US 2007/0027422 A1
(43) Pub. Date: Feb. 1, 2007

(54) Cervical distraction device

(76) Inventors: Carlos Becerra, Atlanta, GA (US); William Keith Chandler, Lawrenceville, GA (US); Scott Powers, Dallas, GA (US); Abebaw Zeleke, Marietta, GA (US)

Correspondence Address: PATTON BOGGS, L.L.P., 2001 ROSS AVENUE, SUITE 3000, DALLAS, TX, 75201, US

(21) Appl. No.: 11/494,325

(22) Filed: Jul. 25, 2006

Related U.S. Application Data
(63) Continuation-in-part of application No. 11/268,181, filed on Nov. 7, 2005.
Application No. 11/268,181 is a continuation of application No. 10/889,422, filed on Jul. 12, 2004, now Pat. No. 6,984,217.
(60) Provisional application No. 60/486,049, filed on Jul. 10, 2003.

(51) Int. Cl. A61F 5/00

(52) U.S. Cl. 602/33

(57) ABSTRACT

A cervical traction device includes a base, a cervical force application member, and a motor operably attached to the cervical force application member. The motor preferably drives the cervical application member through a friction drive system in order to provide a force to a person's cervical vertebrae. The friction drive system provides overload protection to prevent application of excessive traction forces to the patient.

### CROSS-REFERENCE TO RELATED APPLICATIONS

This application is a continuation of U.S. patent application Ser. No. 11/268,181 filed on Nov. 7, 2005, which is a continuation of U.S. patent application Ser. No. 10/889,422 filed on Jul. 12, 2004, now U.S. Pat. No. 6,984,217, which claims the benefit of and priority to U.S. Provisional Application No. 60/486,049, filed Jul. 10, 2003. All of the applications are hereby incorporated by reference.

### BACKGROUND OF THE INVENTION

1. Field of the Invention

The present application relates to cervical traction devices that are used to distract cervical vertebrae for relieving pain and discomfort associated with cervical misalignment and compression.

2. Description of Related Art

Vertebral traction machines and vertebral decompression machines (collectively referred to herein as “vertebral distraction machines”) have been successfully used to treat vertebral misalignment and compression in people suffering mild to severe back pain. By applying a distractive force to the vertebrae, the machines are able to assist in decompressing or realigning the affected vertebrae, thereby relieving the associated pain. Although some machines have been developed for home use, most vertebral distraction machines are operated by a skilled therapist or doctor.

Typically, the vertebral distraction machine includes a system for applying the distractive force to a patient lying on a platform or bed of the machine. In most cases, distraction of the vertebrae in the back is accomplished by attaching a harness to the waist or legs of the patient. The harness is typically connected to either a flexible rope, cable, or webbing, and a force is applied to pull on the lower body of the patient while the upper body remains stationary. The application of force may be accomplished by hanging weights from the rope, cable, or webbing, but it is more common to apply force using a winch that is turned by a clutch-operated motor. The winch is housed in a pedestal at the foot of the bed on which the patient lies, and the therapist directs the application of force by controlling the clutch-operated motor.

Since it is difficult to isolate the cervical vertebrae using lower body harnesses, cervical traction devices have been provided as “add-on” components for vertebral distraction machines. These add-on components typically include a movable head support that is positioned beneath the head of a patient lying on the bed of the distraction machine. The person's head is secured to the movable head support and a force is applied to the head support using ropes, cables, or webbing attached through pulleys to the winch at the pedestal. The primary problem with this method of cervical distraction is that it provides an indirect, flexible power transfer linkage between the motor applying force and the patient's head. This flexible linkage prevents efficient control of the force. Additionally, the forces required for cervical traction are much less than those required for lower vertebral traction; therefore, the conventional motor associated with vertebral distraction machines is oversized and mismatched for applying cervical distraction forces. Some cervical traction devices employ motors positioned nearer to the head of the patient, but these motors are also connected to the patient's head using flexible power transfer equipment such as ropes, cables, and webbing. These devices suffer the same control problems described above.

An additional problem associated with existing cervical traction devices is the unsafe condition that can be created during a power interruption. The clutch-operated motors used with most cervical traction devices completely disengage when power to the motor is interrupted. For a patient undergoing cervical treatment, the rapid relaxation of the cervical distraction force could be painful and cause injury. It would be much preferred to be able to slowly relax the cervical distraction force in the event of a power loss.

A need therefore exists for an improved cervical traction device that eliminates the flexible power transfer equipment associated with existing cervical traction devices. A need further exists for a cervical traction device that does not require use of an outsized and remotely located motor that is used for lower vertebral distraction. Finally, a need exists for a cervical traction device that allows gradual reduction in the cervical distraction force in the event of a power loss or interruption.

### BRIEF SUMMARY OF THE INVENTION

The problems presented by existing cervical traction devices are solved by the systems and methods of the present invention. A cervical traction device is provided in accordance with the principles of the present invention to apply a traction force to a cervical vertebrae of a person. The cervical traction device includes a base and a drive shaft rotatably carried by the base. The drive shaft includes a threadless shaft surface. The cervical traction device further includes a drive block having a shaft channel to receive the drive shaft. At least one bearing is rotatably received by the drive block on a first end of the drive block. The bearing includes a substantially cylindrical drum having a bearing surface to engage the shaft surface. At least one bearing is rotatably received by the drive block on a second end of the drive block opposite the first end. The bearing includes a substantially cylindrical drum having a bearing surface to engage the shaft surface. A cervical force application member is connected to the drive block, and a motor is operably attached to the drive shaft to rotate the drive shaft.

Also in accordance with the principles of the present invention, a cervical traction device is provided that includes a base and a drive shaft rotatably carried by the base. The drive shaft includes a threadless shaft surface. A drive block is provided and includes a shaft channel for receiving the drive shaft. At least one bearing is rotatably received by the drive block on a first end of the drive block. The bearing includes a substantially cylindrical drum having a bearing surface to engage the shaft surface. The cylindrical drum further includes a longitudinal axis that is angled relative to a longitudinal axis of the drive shaft. A cervical force application member is connected to the drive block, and a motor is operably attached to the drive shaft to rotate the drive shaft.

Also in accordance with the principles of the present invention, a cervical traction device is provided that includes a base and at least one bearing mount connected to the base. A drive shaft is rotatably carried by the bearing mount, and the drive shaft includes a threadless shaft surface. A drive block having a first block member and a second block member is provided. At least one of the first and second block members includes a shaft channel, and the first block member is configured to be connected to the second block member such that the shaft passes through the shaft channel. A first plurality of bearings is provided, each bearing being rotatably received by the drive block on a first end of the drive block. Each of the first plurality of bearings includes a substantially cylindrical drum having a bearing surface, and each cylindrical drum of the first plurality of bearings includes a longitudinal axis that is angled relative to a longitudinal axis of the drive shaft. The bearing surface of each of the first plurality of bearings engages the shaft surface when the first and second block members are connected. A second plurality of bearings is also provided, each bearing being rotatably received by the drive block on a second end of the drive block opposite the first end. Each of the second plurality of bearings includes a substantially cylindrical drum having a bearing surface, and each cylindrical drum of the second plurality of bearings includes a longitudinal axis that is angled relative to a longitudinal axis of the drive shaft. The bearing surface of each of the second plurality of bearings engages the shaft surface when the first and second block members are connected. A cervical force application member is connected to the drive block, and a motor is operably attached to the drive shaft to rotate the drive shaft.

Also in accordance with the principles of the present invention, a cervical traction device is provided that includes a cervical force application member adapted to engage a head of a patient. A motor is operably attached to the cervical force application member by a friction drive system to apply a traction force to the cervical force application member.

The above as well as additional objectives, features, and advantages of the present invention will become apparent in the following detailed written description.

### BRIEF DESCRIPTION OF THE DRAWINGS

FIG. 1 illustrates a perspective view of a vertebral distraction apparatus having a cervical traction device according to an embodiment of the present invention mounted thereon;

FIG. 2 depicts a partial side view of the vertebral distraction apparatus of FIG. 1;

FIG. 3 illustrates a front view of the vertebral distraction apparatus of FIG. 1;

FIG. 4 depicts a partial top view of the vertebral distraction apparatus of FIG. 1;

FIG. 5 illustrates a first perspective view of the cervical traction device of FIG. 1;

FIG. 5A depicts a second perspective view of the cervical traction device of FIG. 1;

FIG. 6 illustrates a side view of the cervical traction device of FIG. 1 shown in an elevated position;

FIG. 7 depicts a side view of the cervical traction device of FIG. 1 shown in a non-elevated position;

FIG. 8 illustrates a schematic of the electrical and mechanical connections associated with the cervical traction device of FIG. 1;

FIG. 9 depicts a flow chart showing the steps involved in positioning the cervical traction device of FIG. 1;

FIG. 10 illustrates a flowchart showing a method of decompressing cervical vertebrae according to the present invention;

FIG. 11 depicts a perspective view of a cervical traction device according to an embodiment of the present invention, the cervical traction device having a cervical force application member and a base;

FIG. 12 illustrates a top perspective view of the cervical traction device of FIG. 11, the cervical force application member not being illustrated to more clearly show the base, a drive shaft, and a drive block for driving the cervical force application member;

FIG. 13 depicts a top view of the cervical traction device of FIG. 12;

FIG. 14 illustrates a bottom perspective view of the cervical traction device of FIG. 12;

FIG. 15 depicts a side view of the drive block and drive shaft of FIG. 12, the drive block rotatingly receiving a plurality of bearings;

FIG. 16 illustrates a front view of the drive block and drive shaft of FIG. 15;

FIG. 17 depicts a rear view of the drive block and drive shaft of FIG. 15;

FIG. 18 illustrates a top view of the drive block, drive shaft, and bearings of FIGS. 16 and 17 taken at 20-20, the view further illustrating the angular relationship between each bearing and the drive shaft; and

FIG. 19 depicts a schematic view a fail-safe mechanism operably associated with the cervical traction device of FIG. 11, the fail-safe mechanism having a computer system for monitoring the traction force applied to a patient.

### DETAILED DESCRIPTION OF THE PREFERRED EMBODIMENT

In the following detailed description of the preferred embodiments, reference is made to the accompanying drawings, which form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical software, electrical, mechanical, structural, and material changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

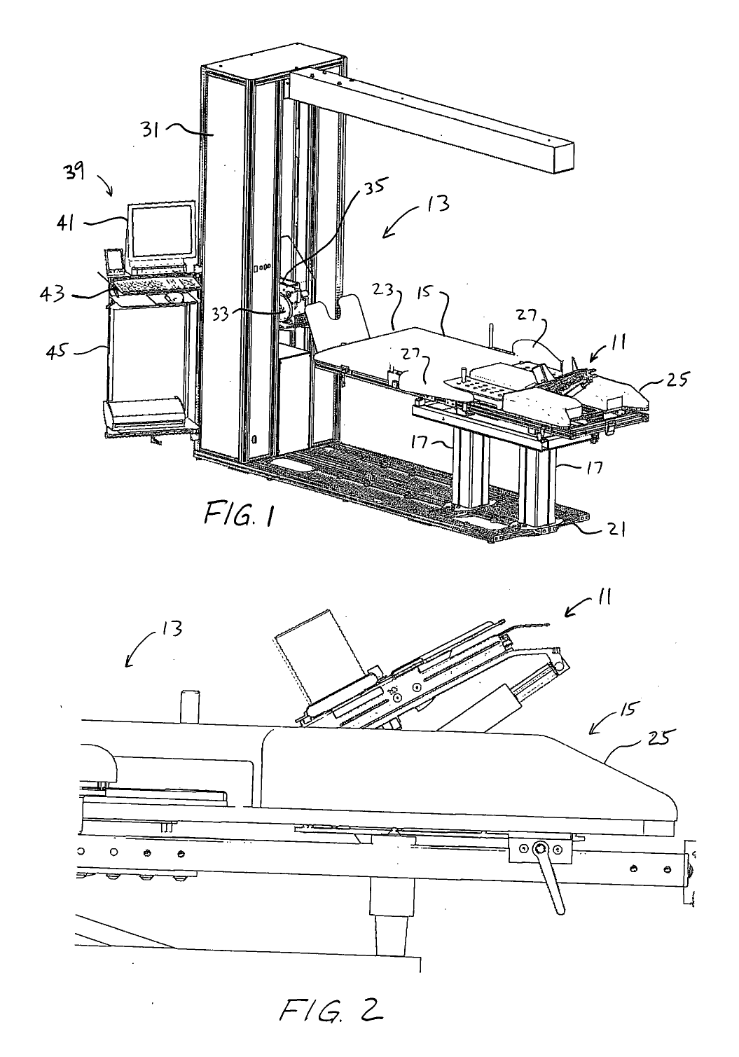

Referring to FIG. 1-4, a cervical traction device 11 according to an embodiment of the present invention is mounted on a conventional vertebral distraction apparatus 13. As referred to herein, the vertebral distraction apparatus 13 should be understood to include any apparatus that typically would be used to apply forces to vertebrae in a person's back, including but not limited to vertebral traction machines and vertebral decompression machines. The vertebral distraction apparatus 13 illustrated in FIGS. 1-4 includes a bed 15 mounted on a plurality of support posts 17, which may be telescopic to allow height adjustment of the bed relative to a floor 19. The support posts 17 terminate at a base plate 21, which further stabilizes the bed 15. Bed 15 includes a first end 23, or foot end, and a second end 25, or head end. When a patient is placed on the bed 15, the feet of the patient are typically oriented toward the first end 23 of the bed, and the head of the patient is typically oriented toward the second end 25 of the bed 15. The bed 15 further includes a pair of armrests 27 for supporting the patient's arms during treatment.

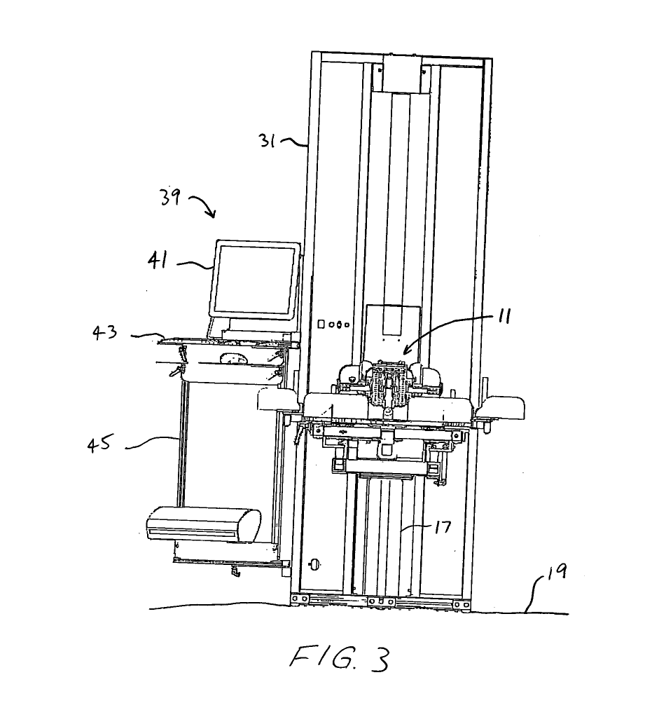

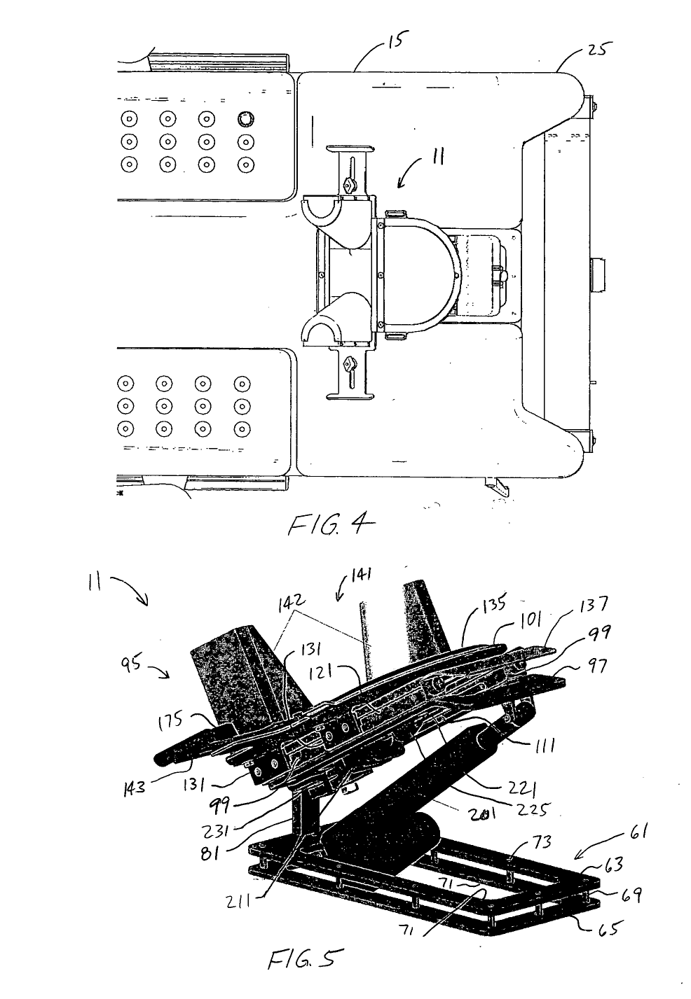

At the first end 23 of bed 15, a pedestal 31, or distraction head, is disposed. The bed 15 may be adjustably attached to the pedestal 31 or may be independently positioned relative to the pedestal 31. Pedestal 31 houses the equipment necessary for applying distraction forces (either traction or decompression forces) to the vertebrae in a person's back. In most instances, pedestal 31 includes a winch 33 having a clutch-operated motor 35. The winch 33 is connected to a harness, strap, rope, or other flexible line (not shown) that can be positioned around the legs or waist of the patient lying on the bed 15. Through the application of force applied by the clutch-operated motor, distraction of the patient's vertebrae, especially the lower vertebrae, is accomplished. Pedestal 31 may also include a computing system 39 having a monitor 41, an input panel 43, or keyboard, a processor (not shown), and memory (not shown). The computing system 39 is used to monitor and/or control the application of force during the distraction of the lower vertebrae and may also store historical data about the particular patient being treated. As best illustrated in FIG. 3, a computer rack 45 may be adjustably connected to the pedestal 31 for supporting the monitor 41. The adjustable connection of the computer rack 45 to the pedestal 31 allows the height of the computer rack 45 to be adjusted to provide easy access by a therapist treating the patient.

The pedestal 31 is typically located near the foot end 23 of the bed 15 so that the motor 35 housed within the pedestal 31 for applying the lower vertebral distraction force is located in close proximity to the lower vertebrae. The pedestal 31 could alternatively be located near the head end 25 of the bed 15. This positioning of the pedestal 31 would require a system of pulleys to route a strap from the head end 25 of the bed 15 to the foot end 23 of the bed to enable application of the distraction forces from the foot end 23 of the bed 15. Alternatively, lower vertebral distraction forces could potentially be applied from the head end 25 of the bed 15 if a harness was connected to the shoulders, arms, or upper body of the patient.

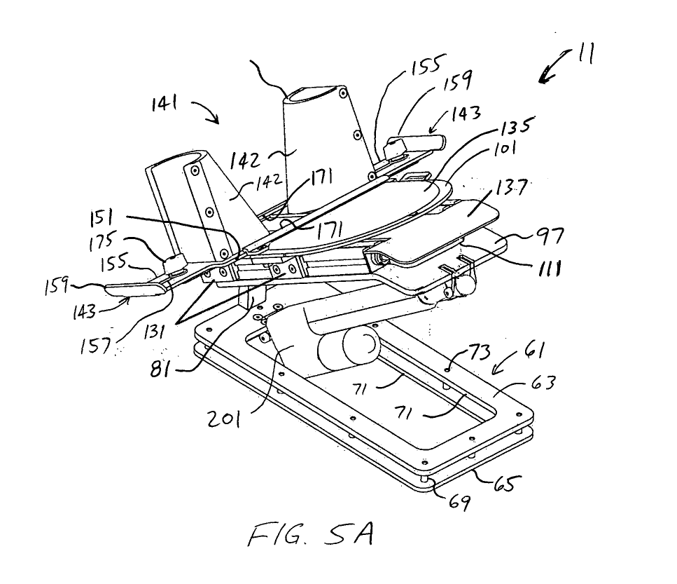

Referring to FIGS. 5-7, the cervical traction device 11 according to an embodiment of the present invention is illustrated independently of the bed 15 in more detail. The cervical traction device 11 generally includes a base 61 having an upper base plate 63 and a lower base plate 65 connected in spaced opposition to one another by a plurality of spacers 69. Each of the base plates 63, 65 includes a center aperture 71, and the spacers 69 are arranged outside of the central apertures 71 near the perimeter of each base plate 63, 65. Preferably, mounting holes 73 pass through each of the base plates 63, 65 and the spacers 69 for mounting the base 61 to the bed 15 or another stabilizing object. Base 61 further includes a stabilization post 81 connected at one end to the upper base plate 63.

An elevation assembly 95 includes an elevation support plate 97, a pair of track members 99, and a cranial support plate 101 and is pivotally connected to the stabilization post 81. Each component of the elevation assembly 95 is meant to be angularly adjustable (or elevated) relative to the base 61, therefore any of these components could be pivotally connected to the stabilization post 81. However, in a preferred embodiment, the elevation support plate 97 is pivotally connected to the stabilization post 81. The elevation support plate 97 includes a central aperture 111, and each of the track members 99 is rigidly connected to an upper surface of the elevation support plate 97 outside of the central aperture 111 in spaced opposition to the other track member 99. Preferably, the track members 99 are extruded from a durable metal material such as steel and include a receiving channel 121. The track members 99 are mounted substantially parallel to one another such that the receiving channels 121 face outward. It is possible, however, to have receiving channels on both sides of the track members 99, or alternatively, to have only one receiving channel 121 per track member 99 and orient the receiving channels 121 inward. The receiving channels 121 are provided to receive bearings and therefore, positioning of the receiving channels 121 (and bearings) farther apart will provide better overall support.

Cranial support plate 101 is slidingly attached to the track members 99 through the use of self-aligning bearings 131. Preferably, four bearing units 131 are rigidly attached to the cranial support plate 101, and the receiving channel 121 on each track member 99 slidingly receives two of the bearing units. The bearings permit movement of the cranial support plate 101 relative to the elevation support plate along an axis parallel to the receiving channels 121. Cranial support plate 101 preferably includes a cushioned head pad 135 for making a patient being treated by the cervical traction device 11 more comfortable. During treatment, the back of the patient's head rests on the head pad 135. A hair guard 137 is rigidly attached to the track members 99 and assists in preventing the patient's hair from becoming entangled in the moving parts of the cervical traction device 11.

The cervical traction device includes a cervical force application member 141 for applying a cervical distraction force to a person's cervical vertebrae. In general, the term “cervical force application member” is used to refer to any of the components that are driven in order to apply the cervical distraction force. In a preferred embodiment, the cervical force application member 141 includes the cranial support plate 101 and a pair of occiput posts 142. Occiput posts 142 are rigidly connected to a pair of occiput positioning plates 143. Each occiput post 142 includes a hemispherical wall 145. The walls 145 of the occiput posts are angularly canted to form a V-shape when the two posts are installed adjacent to one another. The occiput positioning plate 143 is connected to the wall 145 such that a flange 151 extends past the wall on both sides of the occiput post 142. Each occiput positioning plate 143 includes an adjustment region 155 having a slot 157 and a handle region 159. The flanges 151 of the occiput positioning plate 143 are slidingly received by positioning channels 171 on the cranial support plate 101. A thumbscrew 175 is placed through slot 157 and a corresponding slot (not shown) in the cranial support plate 101. By selectively loosening or tightening the thumbscrew 175, each occiput post 142 can be independently adjusted on the cranial support plate 101 in a lateral direction. This allows the distance between the occiput posts 142 to be varied for individual patients.

The occiput posts 142 are the preferred method of applying force to the cervical vertebrae of a patient. The occiput posts 142 are configured to be placed around the patient's neck just beneath the occipital portion of the skull. As force is applied to the occiput posts 142, the force is gently transferred to the head of the patient, thereby minimizing the discomfort that is sometimes associated with cervical distraction. Although the occiput posts 142 are preferred, it should be apparent to a person of ordinary skill in the art that other cervical force application members 141 could be used instead. For example, a pair of lateral support bars could be positioned at the base of the patient's skull and across the patient's chin to apply cervical distraction forces. Alternatively, a harness system could be attached to the cranial support plate 101 for supplying the needed force to the patient's head. Another example could be to have a molded cavity integrally formed on the cranial support plate 101 for surrounding at least a portion of the patient's head.

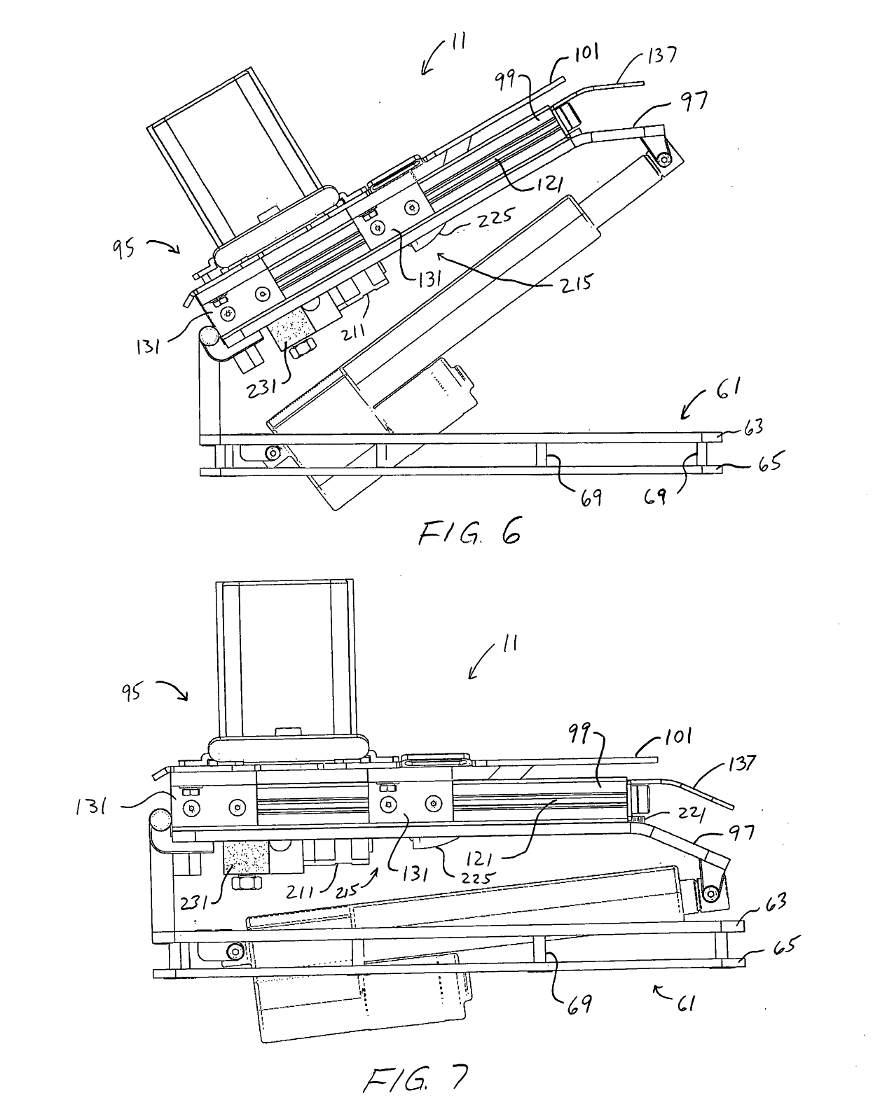

A linear actuator 201 is pivotally connected at one end to the upper base plate 63 and is pivotally connected at a second end to the elevation support plate 97. The linear actuator is positioned within the central apertures 71 of the base plates 63, 65 so that it can operate without obstruction. The linear actuator 201 provides selective positioning of the elevation assembly 95 (the elevation support plate 97, the track members 99, the cranial support plate 101, and the occiput posts 142) either prior to or during treatment of a patient so that the application of force can be properly concentrated on particular areas of the cervical vertebrae. The linear actuator 201 can be adjusted between a fully elevated position (shown in FIG. 6) and a non-elevated position (shown in FIG. 7). Preferably, the non-elevated position would allow the patient's neck to be substantially parallel with the bed 15 at an angle of zero (0) degrees. The fully elevated position preferably positions the patient's neck at an angle of thirty (30) degrees from the surface of the bed 15. Of course, the linear actuator 201 is capable of positioning the elevation assembly 95 at any angle between the non-elevated and fully elevated positions. It is also important to note that while the maximum angle is preferred to be thirty (30) degrees, this design parameter could be increased or decreased. Finally, while it is preferable to use a linear actuator for adjusting the elevation of the cervical traction device 11, the elevation of the device could be positioned manually.

A motor 211 is positioned within the central aperture 111 of the elevation support plate 97 and is rigidly connected to either the elevation support plate 97 or the track members 99. A direct drive system 215 is operably connected between the motor 211 and the cranial support plate 101. For the purposes of the present invention, the phrase “direct drive system” includes any direct, non-flexible linkage between a driving element (e.g. a motor) and a driven element (e.g. cranial support plate 101) that allows a transfer of power between the two elements. Direct drive systems do not include flexible power transfer linkages such as cables, ropes, straps, webbing, or other materials that are typically used with winches and pulleys. The direct drive system 215 according to the present invention preferably includes a threaded shaft 221 rotatably connected to the motor 211. The shaft 221 includes a plurality of threads on its outer surface for threadingly receiving a screw transfer member 225. Screw transfer member 225 is rigidly connected to a lower surface of the cranial support plate 101. As motor 211 turns, shaft 221 turns in response, thereby driving screw transfer member 225 along the shaft 221 in a direction determined by the direction the motor 211 turns. The cranial support plate 101 follows the movement of screw transfer member 225. The motor 211 is therefore capable of applying a force to the cranial support plate 101 and driving the cranial support plate 101 in either of two directions.

Motor 211 is preferably a stepper motor. A stepper motor allows very fine, incremental control over the force applied to the cranial support plate 101 and the resulting movement by the cranial support plate 101. The stepper motor provides controlled application of force in both directions in very small increments. The stepper motor is preferably sized to provide up to fifty (50) pounds of force and provides this force by moving the screw transfer member 225 one hundred and twenty five thousandths (0.0125) of an inch for each step of the motor. Even finer control is provided by using control software, which allows incremental advancement of up to 0.0125/4 inches. In addition to the control advantages provided by the stepper motor, the stepper motor also provides desirable characteristics if power is lost or interrupted during the treatment of a patient. Because of the configuration of the magnets within a stepper motor, a loss of power to the motor does not immediately release all force being applied by the motor. Instead, the force being applied by the motor is relieved slowly in the event of a power loss. This is an important advantage since an instantaneous release of tension from the neck of a patient being treated could cause discomfort and injury. Although the stepper motor is sized to provide up to fifty (50) pounds of force, during most cervical treatments, the force applied by the motor will not exceed 30-40 pounds.

Other types of motors could be used in place of the stepper motor; however, it is desired to maintain good control over the application of force to the cranial support plate 101. An example of another motor type that would satisfy this function includes a servo motor.

A strain gauge 231 is operably connected to the motor 211 to measure the application of force applied by the motor to the cranial support plate 101. Strain gauge 231 is preferably electrically connected to a control system that is discussed in more detail below.

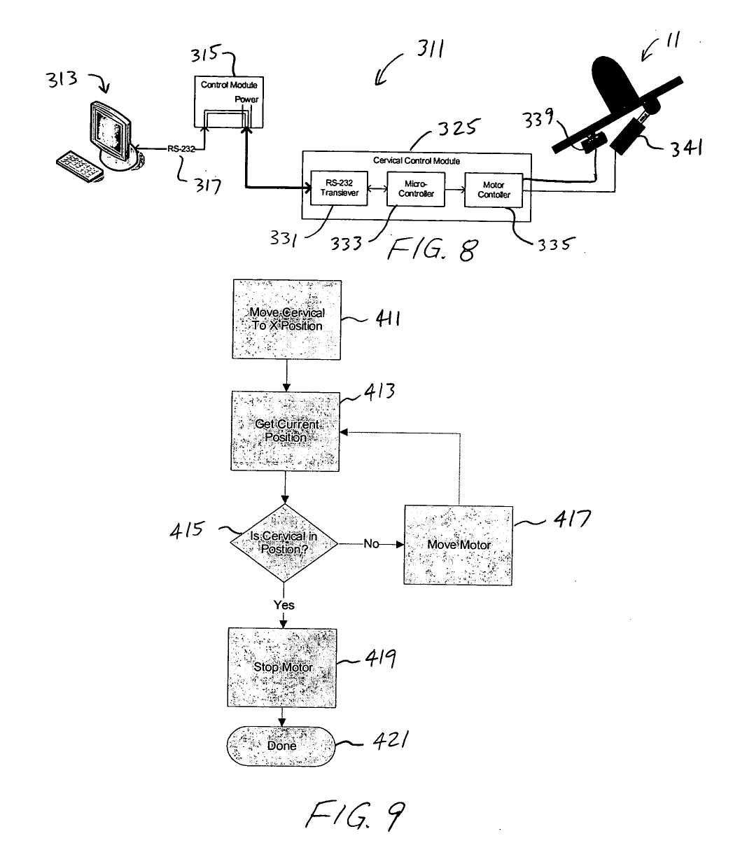

FIG. 8 illustrates a block diagram of an exemplary electrical system 311 for controlling the cervical traction device 11 during treatment of a patient. As shown, a computing system 313 is electrically coupled to a control module 315 via communications bus 317. In one embodiment, the computing system 313 is a conventional personal computer executing a software program with a graphical user interface (GUI) for enabling an operator to establish and/or modify a treatment profile for individual patients. Alternatively, the computing system 313 is an integrated unit having a user interface formed of keypads and an optional display, such as a liquid crystal display, to display the patient treatment profile. When cervical traction device 11 is used with a conventional vertebral distraction machine, computing system 313 will likely be located near pedestal 31 similar to computing system 39 (see FIG. 3). The control module 315 may be located with or separate from the computing system 313. The control module 315 may include a processor (not shown) and transceiver (not shown) for communicating with the computing system 313. The control module 315 is operable to communicate with the computing system 313 for receiving patient profile control commands from the computing system 313. The control module may process the patient profile control commands for communication to a cervical control module 325. The cervical control module 325 may be located at the computing system 313, between the computing system 313 and the cervical traction device 11 (e.g., below the bed 15 of FIG. 1), or at the cervical traction device 11. The communications link between the computing system 313 and cervical control module 325 may be wired or wireless.

The cervical control module 325 may include a communication transceiver 331, micro-controller 333, and motor controller 335. The transceiver 331 may communicate via an RS-232 or other protocol as understood in the art for communicating data in a digital or analog format. The micro-controller 333 may be any micro-controller as understood in the art capable of performing mathematical and logical operations. Alternatively, the micro-controller may be a programmable unit and/or logic circuit that is capable of performing mathematical and logical operations. The micro-controller 333 is operable to execute software or firmware that controls the electro-mechanical operations of the cervical traction device 11 by generating commands and operating in conjunction with the motor controller 335 to control one or more electro-mechanical components 339 and 341 at the cervical traction device 11.

The electro-mechanical components 339 and 341 of the cervical traction device 11 may be a stepper motor and a linear actuator, respectively. The stepper motor may be utilized to apply a force to the head of the patient (similar to motor 211) while the linear actuator may be utilized to adjust the angle of the cervical traction device 11 (similar to linear actuator 201). Although two electro-mechanical components are shown, it should be understood that one or more electro-mechanical components may be utilized to control mechanical operation of the cervical traction device 11 for treating a patient in accordance with the principles of the present invention. As depicted, control of the electro-mechanical components 339 and 341 of the cervical distraction device is performed by communicating one or more signals to the cervical distraction device 11. Again, the signals may be digital or analog as opposed to a mechanical or other force for moving mechanical components at the cervical distraction device 11.

The micro-controller 333 further may be utilized to receive a feedback signal from one or more sensors (such as strain gauge 231 illustrated in FIG. 6) coupled to the cervical distraction device 11. The sensors may be position, speed, strain, and/or acceleration sensors and utilized for enabling the motor-controller 335 to accurately position and move the cervical distraction device 11 in following the patient treatment profile commands generated by the computing system 313. Depending on the feedback provided by the sensors, the micro-controller 333 may also include kill switch functionality to direct the motor-controller 335 to shut down the motor. The kill switch would be activated if sensor values exceed predefined parameters.

In controlling the electro-mechanical components of the cervical distraction device 11, FIG. 9 is a flow diagram describing basic control thereof. The control process starts at step 411. At step 411, the cervical force application member is moved to a first position. At step 413, the actual position is sensed and fed back to determine the actual current position. A determination is made at step 415 if the cervical force application member is at the commanded position. If not, then a correction signal is sent at step 417 to alter the position of the cervical force application member via an electro-mechanical component. Steps 413-417 are repeated until the position is correct. Once the position is correct, the motor is stopped at step 419. The control process ends at step 421.

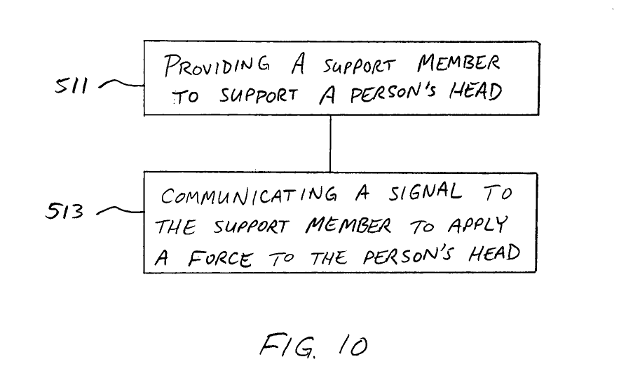

FIG. 10 illustrates a method for distracting a cervical vertebrae according to the present invention. The method includes the steps of providing a support member to support a person's head at step 511 and communicating a signal to the support member to apply a force to the person's head at step 513. The communicated signal is preferably delivered to a stepper motor at the support device, which drives the support device through a direct drive system. The force applied to the person's head may be monitored, and is capable of being gradually decreased.

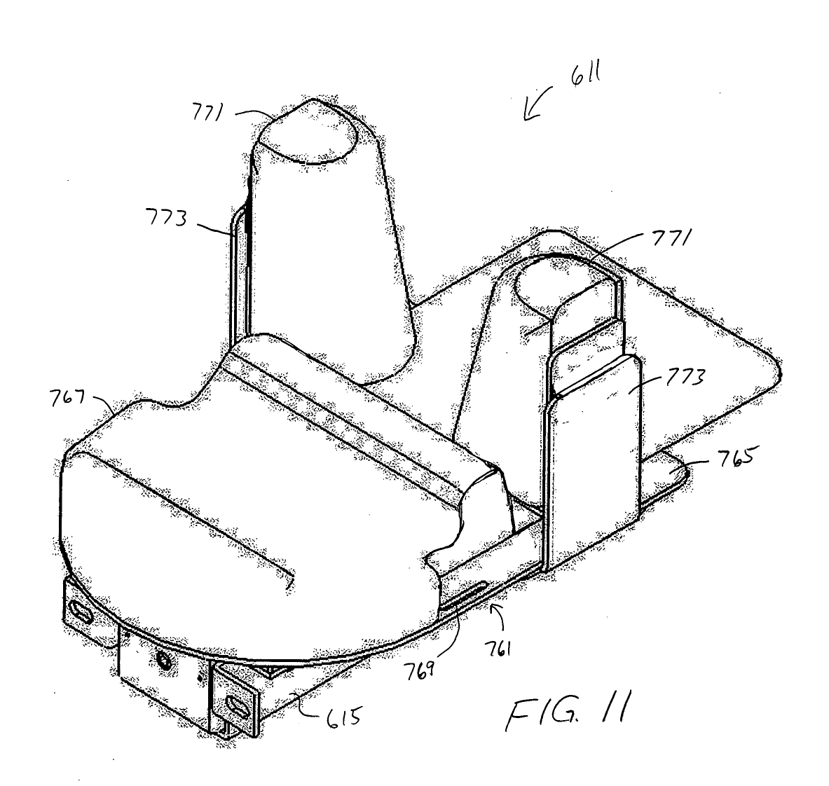

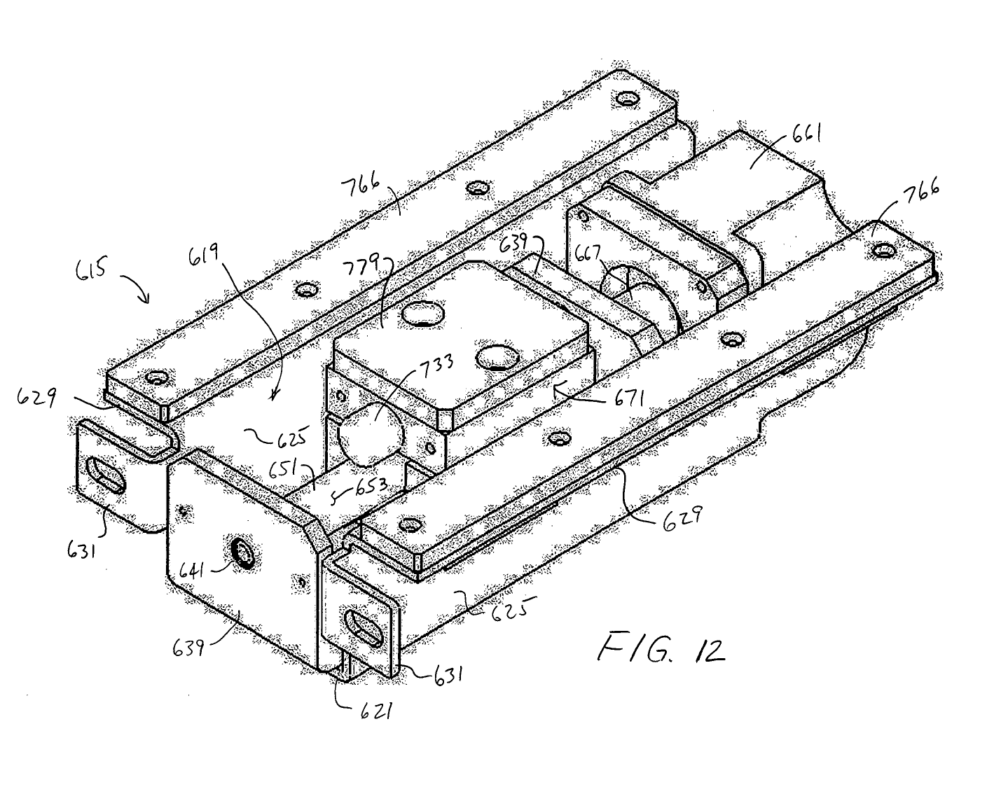

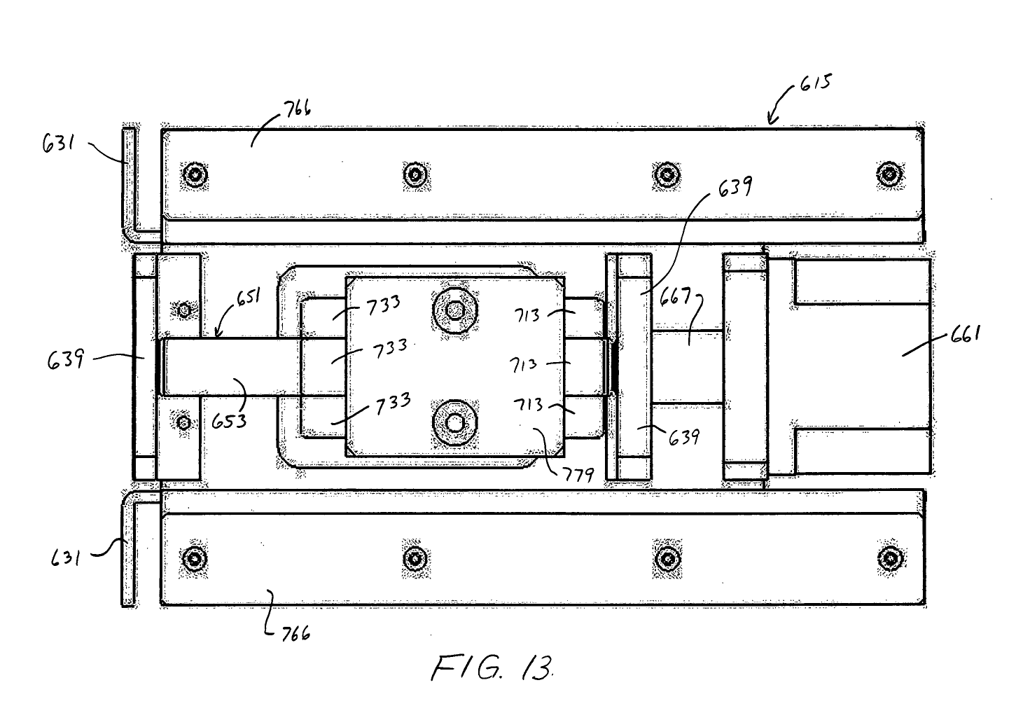

Referring to FIGS. 11-14, a cervical traction device 611 includes a base, or cervical tub 615 positioned beneath a cervical force application member 761. The base 615 includes a basin 619 defined by a floor 621 and a pair of sidewalls 625 integrally connected to the floor 621 on opposite sides of the floor 621. Each sidewall 625 includes a rail 629, the rails 629 being integrally connected to the sidewall 625, and each rail including an upper surface that is substantially planar to the upper surface of the other rail 629. A pair of attachment tabs 631 are integrally connected to the sidewalls 625, and each attachment tab 631 includes an aperture 633 for receiving a bolt or other fastener (not shown) to attach the base 615 to a vertebral distraction apparatus or other structure.

A pair of bearing mounts 639 are connected to the floor 621 of the base 615. Each bearing mount 639 includes an aperture 641. A drive shaft 651 is rotatably carried by the apertures 641 of the bearing mounts 639. The bearing mount 639 may include ball bearings or polymer bushings within the aperture 641 to minimize friction during rotation of the drive shaft 651. It should be noted that the bearing mounts 639 are shown in the illustrated figures as being separate from the base 615; however, the bearing mounts 639 may be integrally formed with the base 615. Similarly, the various portions of the base 615 including the basin 619, the floor 621, the sidewalls 625, the rails 629, and the attachment tabs 631 may be either integrally formed with the other portions of the base 615 or may be separate parts that are attached to one another by welding, adhesives, mechanical fasteners, or other fastening means.

The drive shaft 651 includes a threadless shaft surface 653 and a longitudinal axis about which the drive shaft 651 rotates. The draft shaft 651 is operably coupled to an output shaft 663 of a motor 661 by a coupler 667. Coupler 667 may be a mechanical, magnetic, fluidic or elastomeric coupler, or any other device used to transmit the rotational motion of the output shaft 663 to the drive shaft 651. The motor 661 may be rigidly connected to the floor 621 of the base 615. While the motor 661 may be any device capable of turning the drive shaft 651, motor 661 is preferably a stepper motor. As previously mentioned, a stepper motor allows fine, incremental control over a force that is applied to the output shaft 663 of the motor. Stepper motors also provide controlled application of forces in both directions in very small increments. The stepper motor used with cervical traction device 611 is preferably sized to provide up to about sixty (60) pounds of force by moving the output shaft 663 one hundred twenty-five thousands (0.0125) of an inch for each step of the motor. Finer control can be obtained by using control software, which allows incremental advancement of up to 0.0125/4 inches. In addition to the control advantages provided by the stepper motor, the stepper motor also provides desirable characteristics if power is lost or interrupted during the treatment of a patient. Because of the configuration of the magnets within a stepper motor, a loss of power to the motor does not immediately release all force being applied by the motor. Instead, the force being applied by the motor is relieved slowly in the event of a power loss. This is an important advantage since an instantaneous release of tension from the neck of a patient being treated could cause discomfort and injury. Although the stepper motor is sized to provide up to sixty (60) pounds of force, during most cervical treatments, the force applied by the motor will not exceed thirty to forty (30-40) pounds.

Other types of motors could be used in place of the stepper motor, and it is desired that any motor chosen be capable of maintaining good control over the application of force to the cervical vertebrae of the patient. An example of another motor type that would satisfy this function includes a servo motor. However, a person of ordinary skill in the art will recognize that various types of motors may be suitable.

A friction drive system 671 is provided to transfer power from the draft shaft 651 to the cervical force application member 761. The term “friction drive system” as used herein refers to a drive system that is capable of converting the rotational movement of a drive member (e.g. a drive shaft) into translational movement of a driven member using frictional forces between components of the drive member and the driven member. The drive member may be a drive shaft having a threadless surface. The friction drive system may include a bearing drive system such as the Roh'lix Linear Actuator manufactured by Zero-Max Motion Control Products of Plymouth, Minn. Another example of a friction drive system may include a rolling-ring linear actuator, or any other system that converts rotation movement into translational movement using frictional forces between components of the system.

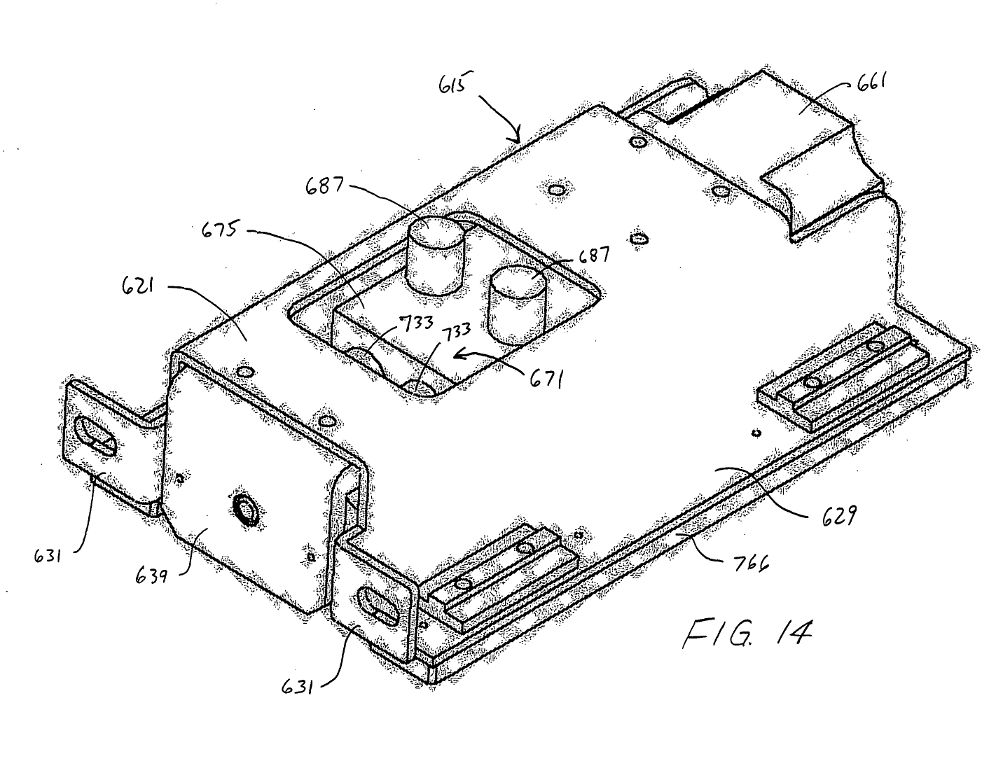

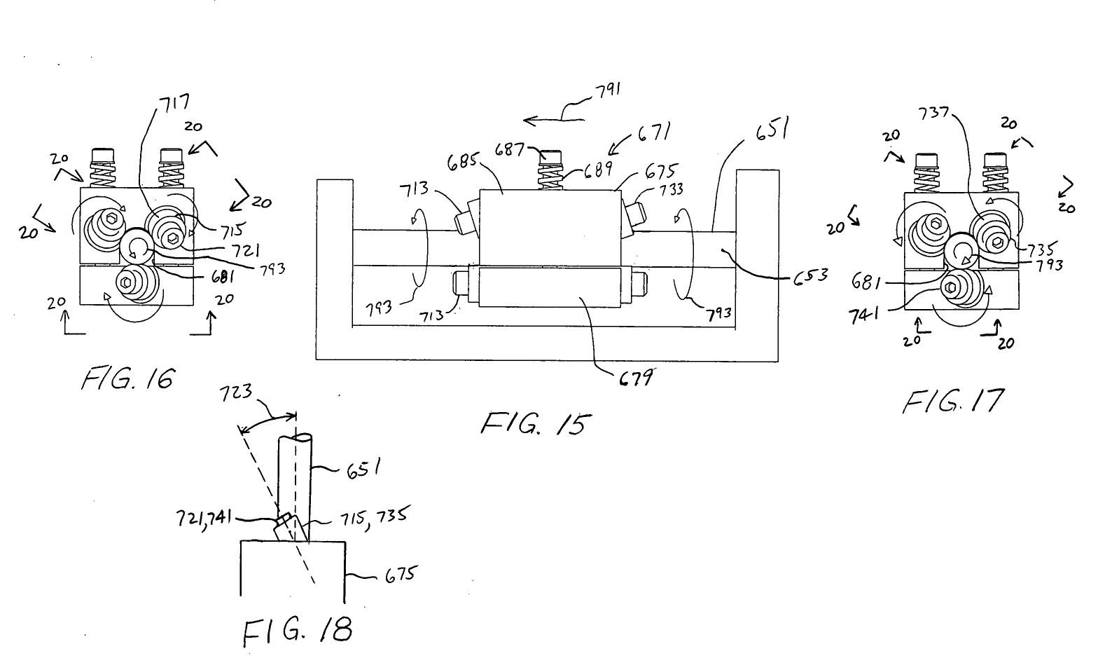

Referring to FIGS. 15-18, in one embodiment, the friction drive system 671 includes a drive block 675 operably associated with a plurality of bearings. The drive block 675 is positioned around the drive shaft 651 and frictional contact between the bearings and the drive shaft 651 allow the rotation of the drive shaft 651 to drive the drive block 675 in translation along the drive shaft 651. The drive block 675 is connected to the cervical force application member 761 to provide a traction force to a cervical vertebrae of a person. In general, the term “cervical force application member” 761 is used herein to refer to any of the components that are driven to apply the cervical traction force. The cervical force application member 761 may include a cranial support plate, or pillow plate 765 that is rigidly connected to the drive block 675. The cranial support plate 765 slidingly engages the base 615 and is supported by the rails 629. A Teflon or other polymer strip 766 may be connected to each rail 629 to provide a reduced friction surface on which the cranial support plate 765 may travel. A pair of strap apertures 769 are provided on opposing sides of the cranial support plate 765 to assist in securing the patient's head during treatment. A cushion 767 is positioned on the cranial support plate 765 to provide comfort and to assist in properly positioning the head and neck of the patient. A pair of occiput posts 771 are connected to the cranial support plate 765 using mounting brackets 773. The occiput posts 771 each include a hemispherical wall that is angularly canted to form a v-shape when the two posts are adjacent one another. The occiput posts 771 may be adjustable as previously explained with respect to occiput post 142 of FIGS. 5-7. A load sensor 779 may be operably connected between the drive block 675 and the cranial support plate 765 to measure the amount of traction force exerted on the patient's cervical vertebrae during treatment.

The drive block 675 may include a first block member 679 and a second block member 685. The second block member 685 preferably includes a shaft channel 681 for receiving the drive shaft 651. It should be noted however that the shaft channel 681 could be disposed alternatively on the first block member 679, or a portion of the shaft channel 681 could be disposed in each of the first and second block members 679, 685. It is also conceivable that a drive block comprised of only one block member may be used; however, it is preferable that a multiple member drive block be used to simplify removal from the drive shaft 651 and to allow adjustments in the maximum thrust transmitted by the drive shaft 651 to the drive block 675. As explained in more detail below, adjustment of the maximum thrust is made possible by the spaced relation of the first and second block members 679, 685 around the drive shaft 651, as well as by thrust adjustment screws 687 and biasing springs 689 connecting the first and second block member 679, 685.

A first plurality of bearings 713 are rotatably received by the drive block 675 in a first end of the drive block 675. Each of the first plurality of bearings 713 includes a substantially cylindrical drum 715 having a bearing surface 717 and a longitudinal axis about which the cylindrical drum 715 rotates. A bearing bolt 721 passes through the cylindrical drum 715 and is used to secure each bearing 713 to the drive block 675. Although the number and spacing of the first plurality of bearings 713 may vary, it is preferred that the first plurality of bearings 713 include three bearings. One of the three bearings may be received by the first block member 679, while the other two bearings are received by the second block member 685. Relative to one another, the bearings 713 are positioned circumferentially around the drive shaft 651 such that the angular spacing 725 between the bearings 713 is equal. When three bearings 713 are used, it is preferred that about one-hundred-and-twenty degrees (120°) of angular spacing be present between each adjacent bearing 713 relative to the center of the drive shaft 651. Each of the bearings 713 is attached to the drive block 675 such that the longitudinal axis of the cylindrical drum 715 is angled relative to the longitudinal axis of the drive shaft 651. The angular relation between the cylindrical drum 715 and the drive shaft 651 is illustrated by angle 723 in FIG. 18. Preferably, angle 723 is approximately 5.625 degrees; however, this angle could be larger or smaller depending on the amount of drive block 675 travel desired for each rotation of the drive shaft 651.

A second plurality of bearings 733 are rotatably received by the drive block 675 in a first end of the drive block 675. Each of the second plurality of bearings 733 includes a substantially cylindrical drum 735 having a bearing surface 737 and a longitudinal axis about which the cylindrical drum 735 rotates. A bearing bolt 741 passes through the cylindrical drum 735 and is used to secure each bearing 733 to the drive block 675. Although the number and spacing of the second plurality of bearings 733 may vary, it is preferred that the second plurality of bearings 733 include three bearings. One of the three bearings may be received by the first block member 679, while the other two bearings are received by the second block member 685. Relative to one another, the bearings 733 are positioned circumferentially around the drive shaft 651 such that the angular spacing 745 between the bearings 733 is equal. When three bearings 733 are used, it is preferred that about one-hundred-and-twenty (120°) of angular spacing be present between each adjacent bearing 733 relative to the center of the drive shaft 651. Each of the bearings 733 is attached to the drive block 675 such that the longitudinal axis of the cylindrical drum 735 is angled relative to the longitudinal axis of the drive shaft 651. The angular relation between the cylindrical drum 735 and the drive shaft 651 is illustrated by angle 723 in FIG. 18. Preferably, angle 723 is approximately 5.625 degrees; however, this angle could be larger or smaller depending on the amount of drive block 675 travel desired for each rotation of the drive shaft 651.

The first and second plurality of bearings 713, 733 cooperate with the biasing springs 689 and the first and second block members 679, 685 to suspend the block members 679, 685 around the drive shaft 651. As mentioned previously, the drive shaft 651 passes through the shaft channel 681, and preferably the drive shaft 651 does not physically contact either the first block member 679 or the second block member 685. Instead, the biasing springs 689 bias the first and second block members 679, 685 apart and assist in positioning the first and second block members 679, 685 in spaced relation to one another as illustrated in FIGS. 15-17. The bearing surfaces 717, 737 of the bearings 713, 733 bear on the shaft surface 653 and further contribute to holding the first and second block members 679, 685 apart. The thrust adjustment screws 687 secure the first and second block members 679, 685 to each other and counteract the biasing force of the biasing springs 689. The thrust adjustment screws 687 exert a force on the first and second block members 679, 685 that causes each bearing 713, 733 to exert a normal force on the drive shaft 651. The normal force may be increased by tightening the thrust adjustment screws 687 to draw the first and second block members 679, 685 closer together. The normal force may be decreased by loosening the thrust adjustment screws 687. A person of ordinary skill in the art of the present invention will recognize that the normal force associated with a particular bearing may be different than the normal force associated with other bearings due to the various positioning of the bearings around the drive shaft 651.

Rotational movement of the drive shaft is converted into translational movement of the drive block 675. Since the bearing surfaces 717, 737 contact the shaft surface 653, frictional forces are transmitted from the shaft surface 653 to the bearing surfaces 717, 737 as the drive shaft 651 is turned by the motor 661. Because of the angled relation of the cylindrical drums 715, 735 to the drive shaft 651, the bearing surfaces 717, 737 each trace a helically shaped footprint along the shaft surface 653 as the drive shaft 651 turns. The angular positioning of the cylindrical drums 715, 735 relative to the drive shaft 651 further results in the frictional forces transferred to the bearing surfaces 717, 737 having a force component that is parallel to the longitudinal axis of the drive shaft 651. This force component is received by the drive block 675 and results in translational movement of the drive block 675 along the drive shaft 651. The angular positioning of the second plurality of bearings 733 is complimentary to the angular positioning of the first plurality of bearings 713 as illustrated in FIGS. 15-17 so that the forces transmitted to the bearings 713, 733 by the drive shaft 651 are complimentary (i.e. do not cancel each other out) for a selected rotation of the drive shaft 651.

Referring still to FIGS. 15-17, the complimentary forces applied to the bearings 713, 733 by the drive shaft 651 result in movement of the drive block 675 in a first translational direction 791 when the drive shaft 651 is rotated in a first rotational direction 793. The drive block 675 moves in a second translational direction (not shown) opposite to the first translational direction 791 when the drive shaft 651 is rotated in a second rotational direction (not shown) opposite to the first rotational direction 793.

The amount of translation distance the drive block 675 travels for each drive shaft 651 revolution is referred to as lead. The lead is determined by the value of the angle 723 (see FIG. 18) between the bearings 713, 733 and the drive shaft 651. Increased angles result in greater distances traveled by the drive block 675 for each revolution of the drive shaft 651. The preferred lead of the drive block 675 is approximately 1/16, although smaller or larger lead values could be selected. For a 1/16 lead, the drive block 675 moves one (1) inch for every sixteen (16) revolutions of the drive shaft 651.

The friction drive system 671 of the cervical traction device 611 provides exceptional safety for a patient undergoing treatment. Since forces are being applied to the patient's vertebrae, it is highly desirable to limit the maximum amount of force that can be applied. If the motor 661 is used to control the maximum application of force (e.g. by providing a motor that can only deliver a certain amount of torque), malfunctions in the motor may cause excessive amounts of force to be exerted on the patient, which could cause severe injury. By using a friction drive system 671 to transmit power between the motor 661 and the patient, the maximum amount of force applied to the patient is more consistently and reliably controlled.

The friction drive system 671 reliably prevents the drive shaft 651 from transmitting more than a selected maximum amount of force. Since power is transmitted from the drive shaft 651 to the drive block 675 by frictional forces, attempts by the motor 661 and drive shaft 651 to transmit forces greater than the selected maximum amount of force result in slippage of the bearings 713, 733 on the drive shaft 651. The drive shaft 651 in this instance may continue to rotate, but the drive block 675 ceases translational motion along the drive shaft 651 due to slippage between the bearing surfaces 717, 737 and the shaft surface 653.

The selected maximum amount of force may be adjusted by tightening or loosening the thrust adjustment screws 687 to increase or decrease, respectively, the normal force that each bearing 713, 733 exerts on the drive shaft 651. Increased normal forces exerted by the bearings 713, 733 result in larger forces that can be transmitted from the drive shaft 651 to the drive block 675 without the bearing surfaces 717, 737 slipping on the shaft surface 653. It follows that decreased normal forces allow the bearing surfaces 717, 737 to slip on the shaft surface 653 in the presence of smaller forces exerted by the drive shaft 651 on the bearings 713, 733.

It is preferred that the selected maximum amount of force for the cervical traction device be set to approximately thirty (30) pounds. The friction drive system could be adjusted to provide as much as sixty (60) pounds of force to the cervical force application member 761, but lower amounts are considered adequate and safer for a wide range of patients. In the event that even higher amounts of force are desired, a friction drive system could be selected that is capable of transmitting higher amounts of force.

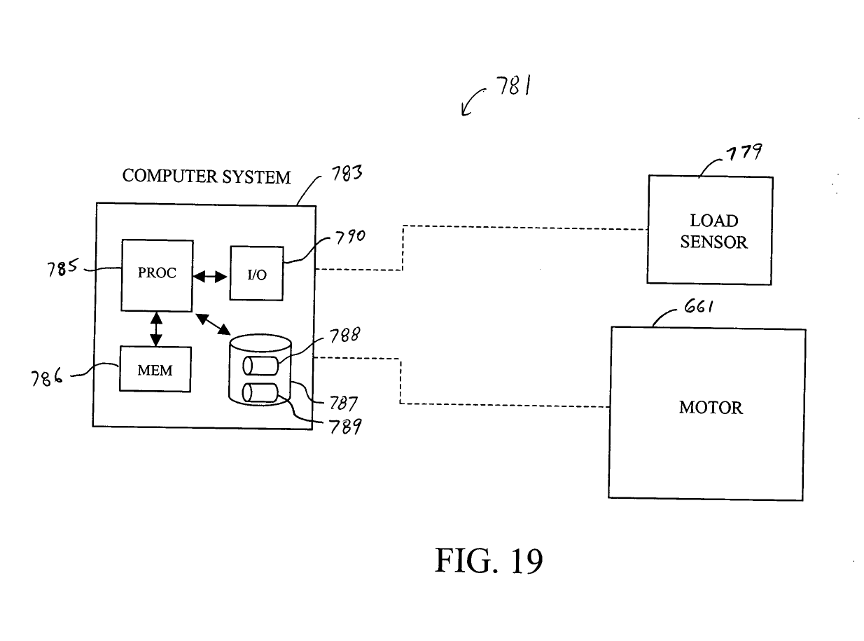

Referring to FIG. 19, in addition to the overload protection provided by the friction drive system 671, the cervical traction device 611 may include a failsafe mechanism 781 for ensuring that excessive traction forces are not exerted on the head or vertebrae of the patient. The failsafe mechanism 781 may include a computer system 783 connected to the load sensor 779. The computer system 783 may include a processor 785 operably connected to a memory medium 786 which may include RAM, ROM, or any other memory medium. The processor 785 may be composed of one or more processors in communication with each other. The computer system 783 further includes a storage device 787 operably connected to the processor 785, the storage device having at least one database 788 or data reservoir and a computer software program 789. The storage device may include a hard drive, magnetic media, optical media, or any other storage medium capable of storing data. The computer system 783 further includes at least one input/output device 790 such as a keyboard, a mouse, or a display monitor.

The processor 785 receives data from the load sensor 779 to determine whether the traction forces being applied to the patient are within an acceptable range of force values. The computer system 783 may be further connected to the motor 661 to shut down power to the motor 661 if the traction forces measured by the load sensor 779 exceed a predetermined force value. The computer software program 789 may further include means for allowing the predetermined force value to be altered. It is preferred that the capability to alter the predetermined force value be password protected to prevent unauthorized adjustment of the predetermined force value and possible injury to a patient.

Referring again to FIGS. 11-18, in operation, a patient that is in need of cervical traction treatment is positioned so that the head of the patient rests upon the cushion 769. The patient is further positioned such that the patient's neck is between the pair of occiput posts and the occiput posts rest just below the jaw of the patient. A strap may be positioned over a portion of the patient's head to further secure the patient to the cranial support plate 765. To treat the patient's cervical vertebrae, the motor 661 is turned on to rotate the drive shaft 651 in the first rotational direction 791. As the drive shaft 651 rotates, the friction drive system 671 coverts the rotational motion of the drive shaft 651 into translational motion of the drive block 675. The drive block 675 moves slowly among the drive shaft 651, driving the cervical force application 761 such that the occiput posts 771 engage the head of the patient. Since the body of the patient is relatively stationary, continued motion of the drive block 675 in the first translational direction 793 increases the traction force applied to the cervical vertebra of the patient. If the motor 661 is stopped at any particular time, the traction force applied at that time remains relatively constant. The traction force may be reduced by reversing the rotation of the motor 661, which results in the drive shaft 651 rotating in the second rotational direction and the drive block 675 moving in the second translational direction. If, while applying traction force, the traction force reaches the selected maximum value of force, any attempt at increasing the traction force will result in slippage of the bearing surfaces 717, 737 on the shaft surface 653, and the drive block 675 will continue to deliver only the selected maximum value of force to the patient. While applying traction force to the patient, the load sensor 779 allows a therapist to monitor the amount of traction force being applied to the patient.

Since the treatment prescribed varies by patient, it may be necessary to increase the selected maximum value of force for certain patients. As previously described, the selected maximum value of force may be increased by tightening the thrust adjustment screws 687. Additionally, the force setting associated with the fail-safe mechanism must also be increased to prevent shutdown of the motor when the load sensor records a higher-than-default traction force.

The cervical traction devices of the present invention presents many advantages over existing equipment used to distract or decompress cervical vertebrae. One advantage is the increased control over the traction or decompression process by providing a direct drive system between the driving component (i.e. a motor) and the driven component (i.e. a cervical force application member). The direct drive system eliminates the need for flexible power transfer devices that are typically used with winches and motors mounted remotely from the patient's head. By mounting the motor for applying the cervical distraction force more closely to the patient's head where the force is to be applied, and by applying the force through the direct drive system, almost no flexibility is introduced between the motor and the person's head, thereby providing a very controlled and efficient application of force. Another advantage is the added overload protection associated with the friction drive system. By using frictional forces to transmit power between drive components, the system can be tuned to prevent the application of traction forces above a selected value.

When mounted on a vertebral distraction apparatus, the motor linked to the cervical traction device is completely separate from the motor traditionally used with the vertebral distraction apparatus to provide lower vertebral distraction forces. As mentioned above, the inclusion of this additional motor allows much more control over the forces applied to the cervical vertebrae. The force control of the cervical traction device is further enhanced by the use of a stepper motor sized specifically for providing cervical distraction forces. The inherent design of a stepper motor allows the gradual and controlled application of force. Since the stepper motor is dedicated to the cervical traction device, it can be much smaller than the traditional motor used to apply lower vertebral distraction forces. Finally, utilizing a stepper motor in the distraction system design allows the application of force to be gradually disengaged in the event of a power interruption to the motor.

Although many of the examples discussed herein are applications of the present invention with conventional vertebral distraction machines, the cervical traction device can be used independently of such machines or can be integrated into new vertebral distraction machines. The cervical traction devices could also be integrated onto an existing or specially designed chair for providing cervical traction when a patient is sitting or reclining. It should further be appreciated that the materials used to construct the cervical distraction device could vary, but preferably include materials having sufficient strength to adequately transmit distraction forces to a patient's cervical vertebrae. It should further be appreciated that certain movable components of the cervical traction device, such as the occiput posts, can be manually adjusted as explained herein, or could be automatically adjusted using motors and sensors. Finally, it should be appreciated that the control, application, and monitoring of force by the cervical traction device may be controlled by a software program associated with the computing system previously discussed. This program may provide simple control of the cervical traction device, thereby enabling a therapist to “dial in” a particular force, or may include a plurality of pre-established or custom routines that apply varying forces over varying time periods to the patient.

It should be apparent from the foregoing that an invention having significant advantages has been provided. While the invention is shown in only a few of its forms, it is not just limited but is susceptible to various changes and modifications without departing from the spirit thereof.

We claim:

1. A cervical traction device for applying a traction force to a cervical vertebrae of a person, the device comprising:
a base;
a drive shaft rotatably carried by the base, the drive shaft having a threadless shaft surface;
a drive block having a shaft channel to receive the drive shaft;
at least one bearing rotatably received by the drive block on a first end of the drive block, the at least one bearing including a substantially cylindrical drum having a bearing surface to engage the shaft surface;
at least one bearing rotatably received by the drive block on a second end of the drive block opposite the first end, the at least one bearing including a substantially cylindrical drum having a bearing surface to engage the shaft surface;
a cervical force application member connected to the drive block; and
a motor operably attached to the drive shaft to rotate the drive shaft.

2. The cervical traction device according to claim 1, wherein the cervical force application member further comprises:
a cranial support plate; and
a pair of occiput posts mounted to the cranial support plate.

3. The cervical traction device according to claim 1, wherein the cylindrical drums of the bearings each include a longitudinal axis that is angled relative to a longitudinal axis of the drive shaft.

4. The cervical traction device according to claim 3, wherein a footprint of the bearing surfaces on the shaft surface as the drive shaft rotates is helical in shape due to the angled positioning of the bearings relative to the drive shaft.

5. The cervical traction device according to claim 1, wherein:
the rotation of the drive shaft in a first rotational direction imparts forces to the bearing surfaces of the bearings to drive the drive block along the drive shaft in a first translational direction parallel to the longitudinal axis of the drive shaft; and
the cervical force application member moves with the drive block to apply the traction force to the cervical vertebrae of the person.

6. The cervical traction device according to claim 5, wherein:
the rotation of the drive shaft in a second rotational direction opposite to the first rotation direction imparts forces to the bearing surfaces of the bearings to drive the drive block along the drive shaft in a second translational direction opposite to the first translational direction and parallel to the longitudinal axis of the drive shaft; and
the cervical force application member moves with the drive block to remove the traction force from the cervical vertebrae of the person.

7. The cervical traction device according to claim 1 further comprising a load sensor operably connected between the drive block and cervical force application member to measure the traction force applied to the person's cervical vertebrae by the cervical force application member.

8. The cervical traction device according to claim 7 further comprising a fail-safe mechanism to cease power to the motor if the traction force measured by the load sensor exceeds a predetermined force value.

9. The cervical traction device according to claim 8, wherein the fail-safe mechanism includes a processor and computer software.

10. The cervical traction device according to claim 1, wherein the motor is a stepper motor.

11. The cervical traction device according to claim 1, wherein the motor is a servo motor.

12. The cervical traction device according to claim 1 further comprising a linear actuator for elevating the cervical force application member.

13. The cervical traction device according to claim 1 further comprising:
a linear actuator for elevating the cervical force application member; and
wherein the linear actuator is capable of elevating the cervical force application member between 0 and 30 degrees from the base.

14. A cervical traction device for applying a traction force to a cervical vertebrae of a person, the device comprising:
a base;
a drive shaft rotatably carried by the base, the drive shaft having a threadless shaft surface;
a drive block having a shaft channel to receive the drive shaft;
at least one bearing rotatably received by the drive block on a first end of the drive block, the at least one bearing including a substantially cylindrical drum having a bearing surface to engage the shaft surface, the cylindrical drum having a longitudinal axis that is angled relative to a longitudinal axis of the drive shaft;
a cervical force application member connected to the drive block; and
a motor operably attached to the drive shaft to rotate the drive shaft.

15. The cervical traction device according to claim 14, wherein the cervical force application member further comprises:
a cranial support plate; and
a pair of occiput posts mounted to the cranial support plate.

16. The cervical traction device according to claim 14, wherein a footprint of the bearing surface on the shaft surface as the drive shaft rotates is helical in shape due to the angled positioning of the at least one bearing relative to the drive shaft.

17. The cervical traction device according to claim 14, wherein:
the rotation of the drive shaft in a first rotational direction imparts a force to the bearing surface of the at least one bearing to drive the drive block along the drive shaft in a first translational direction parallel to the longitudinal axis of the drive shaft; and
the cervical force application member moves with the drive block to apply the traction force to the cervical vertebrae of the person.

18. The cervical traction device according to claim 17, wherein:
the rotation of the drive shaft in a second rotational direction opposite to the first rotation direction imparts a force to the bearing surface of the at least one bearing to drive the drive block along the drive shaft in a second translational direction opposite to the first translational direction and parallel to the longitudinal axis of the drive shaft; and
the cervical force application member moves with the drive block to remove the traction force from the cervical vertebrae of the person.

19. The cervical traction device according to claim 14 further comprising a load sensor operably connected between the drive block and cervical force application member to measure the traction force applied to the person's cervical vertebrae by the cervical force application member.

20. The cervical traction device according to claim 19 further comprising a fail-safe mechanism to cease power to the motor if the traction force measured by the load sensor exceeds a predetermined force value.

21. The cervical traction device according to claim 20, wherein the fail-safe mechanism includes a processor and computer software.

22. A cervical traction device comprising:
a base;
at least one bearing mount connected to the base;
a drive shaft rotatably carried by the bearing mount, the drive shaft having a threadless shaft surface;
a drive block having a first block member and a second block member, at least one of the first and second block members including a shaft channel, the first block member being configured to be connected to the second block member such that the shaft passes through the shaft channel;
a first plurality of bearings, each bearing rotatably received by the drive block on a first end of the drive block, each of the first plurality of bearings including a substantially cylindrical drum having a bearing surface, each cylindrical drum of the first plurality of bearings having a longitudinal axis that is angled relative to a longitudinal axis of the drive shaft, the bearing surface of each of the first plurality of bearings engaging the shaft surface when the first and second block members are connected;
a second plurality of bearings, each bearing rotatably received by the drive block on a second end of the drive block opposite the first end, each of the second plurality of bearings including a substantially cylindrical drum having a bearing surface, each cylindrical drum of the second plurality of bearings having a longitudinal axis that is angled relative to a longitudinal axis of the drive shaft, the bearing surface of each of the second plurality of bearings engaging the shaft surface when the first and second block members are connected;
a cervical force application member connected to the drive block; and
a motor operably attached to the drive shaft to rotate the drive shaft.

23. The cervical traction device according to claim 22, wherein the cervical force application member further comprises:
a cranial support plate; and
a pair of occiput posts mounted to the cranial support plate.

24. The cervical traction device according to claim 22, wherein the rotation of the drive shaft in a first rotational direction imparts forces to the bearing surfaces of the first and second plurality of bearings to drive the drive block and cervical force application member in a first translational direction parallel to the longitudinal axis of the drive shaft.

25. The cervical traction device according to claim 24, wherein the rotation of the drive shaft in a second rotational direction opposite to the first rotation direction imparts forces to the bearing surfaces of the first and second plurality of bearings to drive the drive block and cervical force application member in a second translational direction opposite to the first translational direction and parallel to the longitudinal axis of the drive shaft.

26. The cervical traction device according to claim 22 further comprising a load sensor operably connected between the drive block and cervical force application member to measure a traction force applied to a person's cervical vertebrae by the cervical force application member.

27. The cervical traction device according to claim 26 further comprising a fail-safe mechanism to cease power to the motor if the traction force measured by the load sensor exceeds a predetermined force value.

28. The cervical traction device according to claim 27, wherein the fail-safe mechanism includes a processor and computer software.

29. The cervical traction device according to claim 22, wherein:
each of the first plurality of bearings is circumferentially spaced around the drive shaft such that an angular spacing between adjacent bearings of the first plurality of bearings is equal; and
each of the second plurality of bearings is circumferentially spaced around the drive shaft such that an angular spacing between adjacent bearings of the second plurality of bearings is equal.

30. The cervical traction device according to claim 29, wherein:
the first plurality of bearings includes three bearings;
the second plurality of bearing includes three bearings; and
the angular spacings between adjacent bearings of the first and second plurality of bearings is about 120 degrees.

31. The cervical traction device according to claim 22, wherein a footprint of the bearing surfaces on the shaft surface as the drive shaft rotates is helical in shape due to the angled positioning of the first and second plurality of bearings relative to the drive shaft.

32. The cervical traction device according to claim 22, wherein contact between the bearing surfaces and the shaft surface of the drive shaft suspends the drive block such that the drive shaft does not contact the first or second block members.

33. The cervical traction device according to claim 22, wherein the motor is a stepper motor.

34. The cervical traction device according to claim 22, wherein the motor is a servo motor.

35. The cervical traction device according to claim 22 further comprising a linear actuator for elevating the cervical force application member.

36. The cervical traction device according to claim 22 further comprising:
a linear actuator for elevating the cervical force application member; and
wherein the linear actuator is capable of elevating the cervical force application member between 0 and 30 degrees from the base.

37. A cervical traction device comprising:
a cervical force application member adapted to engage a head of a patient; and
a motor operably attached to the cervical force application member by a friction drive system to apply a traction force to the cervical force application member.

38. The cervical traction device according to claim 37, wherein the motor is a stepper motor.

39. The cervical traction device according to claim 37, wherein the motor is a servo motor.

40. The cervical traction device according to claim 37 further comprising a linear actuator for elevating the cervical force application member.

41. The cervical traction device according to claim 37 further comprising:
a linear actuator for elevating the cervical force application member; and
wherein the linear actuator is capable of elevating the cervical force application member between 0 and 30 degrees from the base.

42. The cervical traction device according to claim 37, wherein the friction drive system further comprises:
a shaft having a threadless shaft surface, the shaft rotatably connected to the base; and
a drive block having at least one rotatable bearing, the bearing having a longitudinal axis about which the bearing rotates and a bearing surface that engages the shaft such that the longitudinal axis of the bearing is angled relative to a longitudinal axis of the shaft.

43. The cervical traction device according to claim 37, wherein the friction drive system is a rolling-ring actuator.

44. The cervical traction device according to claim 37 further comprising a strain gauge operably connected to the cervical force application member to measure the traction force.

45. The cervical traction device according to claim 37, wherein the cervical force application member further comprises:
a cranial support plate; and
a pair of occiput posts adjustably mounted to the cranial support plate.
